(19) United States
(12) Patent Application Publication
(10) Pub. No.: US 2012/0021431 A1
(43) Pub. Date: Jan. 26, 2012

(54) DIAGNOSTIC METHODS USING BNP

(75) Inventors: Toshio Nishikimi, Kyoto-shi (JP); Naoto Minamino, Osaka (JP)

(73) Assignees: Toshio Nishikimi, Kyoto-shi (JP); PRESIDENT OF NATIONAL CARDIOVASCULAR CENTER, Osaka (JP); SHIONOGI & CO., LTD., Osaka (JP)

(21) Appl. No.: 13/035,327

(22) Filed: Feb. 25, 2011

Related U.S. Application Data
(60) Provisional application No. 61/308,034, filed on Feb. 25, 2010.

(51) Int. Cl. G01N 33/74; C07K 16/26

(52) U.S. Cl. 435/7.1; 530/389.2

(57) ABSTRACT

The present invention provides a new method and kit for determining the overload of atrium or ventricle in a subject comprising at least a step of measuring levels of proBNP-108 in a sample from the subject. The disclosed methods and kits are useful, for example, in the diagnosis, prevention and/or treatment of cardiac diseases, particularly heat failure, aortic stenosis, aortic regurgitation, mitral stenosis, mitral regurgitation, and atrial fibrillation.

### CROSS-REFERENCE TO RELATED APPLICATIONS

This application claims the benefit of U.S. Provisional Patent Application Ser. No. 61/308,034, filed Feb. 25, 2010.

The Sequence Listing associated with this application is provided in text format in lieu of a paper copy, and is hereby incorporated by reference into the specification. The name of the text file containing the Sequence Listing is Sequence_Listing.txt. The text file is 2,847 bytes in size, was created on Feb. 25, 2011, and is being submitted electronically via EFSWeb.

### BACKGROUND OF THE INVENTION

1. Field of the Invention

The present invention relates to a medicine for the heart, more preferably, a novel diagnostic method of heart disease.

2. Related Art

Brain natriuretic peptide (BNP) is a cardiac hormone and a member of the natriuretic peptide family (THE HANDBOOK OF BIOLOGICALLY ACTIVE PEPTIDES: Academic Press, 2006, pp. 1217-1225). BNP has a striking similarity to atrial natriuretic peptide (ANP) with regard to both its amino acid sequence and its pharmacologic property (Nature 1988 Mar. 3; Vol. 332: pp. 78-81). ANP is mainly produced and secreted in atrium, whereas BNP is mainly produced and secreted in ventricle (Cardiovasc. Res. 2006; Vol. 69: pp. 318-28). Ventricular wall stress and ischemia stimulate BNP gene expression in the ventricle, and proBNP[1-108] (proBNP-108) is produced in the heart (J. Am. Coll. Cardiol. 2007; Vol. 50: pp. 2357-68 and Heart 2006; Vol. 92: pp. 843-9). When proBNP-108 is secreted from the ventricular myocyte (J. Am. Coll. Cardiol. 2007; Vol. 50: pp. 2357-68 and Heart 2006; Vol. 92: pp. 843-9), it is thought to be cleaved to proBNP[77-108] (BNP-32) and N-terminal proBNP[1-76] (NT-proBNP) in an equimolar fashion by furin, which is a type of endoprotease and prohormone converting enzyme. Especially, both BNP-32 and NT-proBNP are useful as diagnostic marker for heart diseases. Therefore, antibodies thereto and assay method for detecting the same materials have been developed (THE HANDBOOK OF BIOLOGICALLY ACTIVE PEPTIDES: Academic Press, 2006, pp. 1217-1225; Nature 1988 Mar. 3; Vol. 332: pp. 78-81; and Cardiovasc. Res. 2006; Vol. 69: pp. 318-28).

However, recent studies have shown that not only BNP-32 and NT-proBNP-76, but also proBNP-108 circulates in human plasma and that the level of proBNP-108 is also increased upon heart failure (J. Am. Coll. Cardiol. 2007; Vol. 49: pp. 1193-202; J. Am. Coll. Cardiol. 2008; Vol. 51: pp. 1874-82; and Clin. Chem. 2007; Vol. 53: pp. 866-73). Other studies have found that the conventional measurement kit for BNP-32 exhibited proBNP-108 at high cross-reactivity between authentic BNP-32 and proBNP-108 (Clin. Chem. 2007; Vol. 53: pp. 866-73 and Clin. Chem. Acta 2003; Vol. 334: pp. 233-9).

Moreover, the reason why proBNP-108 is secreted without specific conversion into BNP-32 and NT-proBNP-76 remains unknown. The proBNP-108 in plasma from patients with heart failure has not been well investigated. In addition, plasma proBNP-108 is very recently reported to be subjected to O-glycosylation at the N-terminus of the peptide (J. Am. Coll. Cardiol. 2007; Vol. 49: pp. 1071-8 and Biochem. Biophys. 2006; Vol. 451: pp. 160-6).

### SUMMARY OF THE INVENTION

In one aspect, the present invention provides a method for determining the overload of either atrium or ventricle comprising the step of measuring proBNP-108 in a sample from a subject.

In another aspect, the present invention provides a method for determining levels of progressing or treatment effect of heart failure comprising the step of measuring levels of proBNP-108 in a sample from a subject.

In another aspect, the present invention provides a kit for determining the overload of either atrium or ventricle comprising a substance that specifically binds to proBNP-108.

In another aspect, the present invention provides a kit for determining levels of progressing or treatment effect of heart failure comprising a substance that specifically binds to proBNP-108.

These and other aspects of the present invention will become apparent upon reference to the following detailed description. All references disclosed herein are hereby incorporated by references in their entirely as if each was incorporated individually.

### BRIEF DESCRIPTION OF THE DRAWING

FIG. 1 depicts molecular forms of BNP obtained from gel filtration HPLC of the venous plasma extracts from normal subject (A), patient with atrial fibrillation (B) and patient with heart failure (C). Two peaks of high MW and low MW IR-BNP corresponding to IR-proBNP-108, which is high molecular weight immunoreactive (IR-) BNP including both proBNP-108 and glycosylated pro BNP108, and IR-BNP-32, which is a low molecular IR-BNP mainly consisting of BNP-32, were observed. Arrows indicate 1: void volume, 2: glycosylated proBNP-108, 3: proBNP-108, and 4: BNP-32. Sodium chloride (NaCl) was eluted at fraction 40 (not shown in this figure).

FIG. 2 depicts the proBNP-108/total BNP ratios in the normal subject, patient with atrial fibrillation (Af) and patient with heart failure (HF). Data are shown in mean±SD.

FIG. 3 depicts the correlation between plasma IR-proBNP-108 and IR-BNP-32. The scales in both axes are pg per ml.

FIG. 4 depicts BNP molecular forms in extracts of gel filtration HPLC of venous plasma of patents with heart failure with atrial overload (A) and ventricular overload (B). Arrows indicate 1: void volume, 2: glycosylated proBNP-108, 3: proBNP-108, and 4: BNP-32.

FIG. 5 depicts the proBNP-108/total BNP ratio in the atrial overload and ventricular overload groups. Data are shown in mean±SD.

FIG. 6 depicts molecular forms of BNP in gel filtration HPLC extracts of atrial (A) and ventricular (B) tissues of patients with heart failure who have undergone cardiac surgery. Arrows indicate 1: void volume, 2: glycosylated proBNP-108, 3: proBNP-108, and 4: BNP-32.

FIG. 7 depicts the proBNP-108/total BNP ratio in the atrial and ventricular tissues. Data are shown in mean±SD.

FIG. 8 depicts molecular forms of BNP in gel filtration HPLC extracts of pericardial fluid and plasma obtained from heart failure patients with atrial overload (A, B) and ventricular overload (C, D) who have undergone cardiac surgery. A and C indicate the data of the plasma extracts, B and D indicate those of the pericardial fluid. Arrows indicate 1: void volume, 2: glycosylated proBNP-108, 3: proBNP-108, and 4: BNP-32.

FIG. 9 depicts the proBNP-108/total BNP ratios in pericardial fluid and plasma from the 8 patients. Data are shown in mean±SD.

FIG. 10 depicts the relationships between total IR-BNP and proBNP-108/total BNP ratio in patients with heart failure when the heart failure condition changes. Panel A shows correlated negative changes in the proBNP-108/total BNP ratios and total IR-BNP concentrations in the plasma from the same patients with decompensate heart failure obtained before (closed circle) and after (open circle) treatments; Panel B shows correlated positive changes in the proBNP-108/total BNP ratios and total IR-BNP concentrations in the plasma from the same patients before (closed circle) and after (open circle) deterioration of heart failure.

### DETAILED DESCRIPTION OF THE INVENTION

Hereinafter the present invention is described. It should be understood throughout the present specification that expression of a singular form includes the concept of the plurality thereof unless otherwise mentioned. Specifically, articles for a singular form (e.g., “a”, “an”, “the”, etc. in the English language) include the concept of the plurality thereof unless otherwise mentioned. It should be also understood the terms as used herein have definitions typically used in the art unless otherwise mentioned. Thus, unless otherwise defined, all scientific and technical terms have the same meanings as those generally used by those skilled in the art to which the present invention pertains. If there is contradiction, the present specification (including the definition) takes precedence.

### DEFINITIONS

Terms particularly used herein are defined as follows.

As used herein, the term “proBNP-108” refers to precursor of BNP, typically consisting of 108 amino acids (SEQ ID:2). proBNP-108 is produced in the heart when ventricular wall stress and ischemia stimulate BNP gene expression in the ventricle. Moreover, the term “proBNP-108” includes glycosylated proBNP-108. After secreted from the ventricular myocyte, proBNP-108 is known to be cleaved to proBNP [77-108] (BNP-32) and N-terminal proBNP [1-76] (NT-proBNP-76) in an equimolar amount by protease. It is also known that proBNP-108 circulates in human plasma (J. Am. Coll. Cardiol. 2007; Vol. 50: pp. 2357-68, and Heart 2006; Vol. 92: pp. 843-9) and level of this peptide also increases in patient with heart failure (J. Am. Coll. Cardiol. 2007; Vol. 49: pp. 1193-202; J. Am. Coll. Cardiol. 2008; Vol. 51: pp. 1874-82; and Clin. Chem. 2007; Vol. 53: pp. 866-73).

As used herein, the term “BNP-32” refers to peptide consisting of amino acid sequence at position from 77 through 108 in proBNP-108 (SEQ ID NO:3). BNP-32 is known to circulate in human plasma (J. Am. Coll. Cardiol. 2007; Vol. 49: pp. 1193-202; J. Am. Coll. Cardiol. 2008; Vol. 51: pp. 1874-82; and Clin. Chem. 2007; Vol. 53: pp. 866-73). Moreover, BNP-32 is often simply described as “BNP”. In this description, all isoform and splice variants are included in this term.

As used herein, the term “total BNP” refers to the entirety of BNP-32, its precursor, and any cleaved products. “Total BNP” may be measured by measuring each molecular form, such as BNP-32 and proBNP-108 and their combining results. Additionally, for example, antibodies recognizing the common region thereof (e.g. region of 77-108) may be used to measure “total BNP”. These antibodies are commercial available and may be readily obtained, such as SHIONORIA BNP KIT (SHIONOGI&CO., LTD)

As used herein, the term “proBNP-108/total BNP” refers to the ratio of proBNP-108 to total BNP.

As used herein, the term “sample derived from subject” refers to a portion of body from a subject expected to include proBNP-108, BNP-32 and the like. The term includes, but is not limited to, for example, pericardial effusion, blood, and processed blood (for example, plasma, serum, etc.). To carry out more accurate diagnosis, various samples obtained from subject's body, such as tissue, cells and fluid (for example, brain fluid, lymph fluid) may be employed.

As used herein, the term “subject” refers to an organism which is treated according to the present invention, also called “patient”. A patient or a subject may be preferably a human.

As used herein, the term “sample derived from peripheral blood” refers to sample derived from peripherals blood in subjects and its component. The term includes, but is not limited to, for example, peripheral blood plasma and platelet fractions. Sample derived from peripheral blood may be obtained from subjects according to DSM-IV (Diagnostic and Statistical Manual of Mental Disorders, the 4th Ed.).

As used herein, the term “pericardial effusions”, which is also called “pericardial cavity fluid”, refers to the liquid stored between the heart and pericardium which is sac covering the heart.

As used herein, the term “overload” to either atrium or ventricle refers to any overload of subject (herein either atrium or ventricle) and typically refers to increase in pressure or volume (blood volume). Typical examples of causes of such overload, may include damage, disorder, disease of the subject and elevation of blood pressure in the whole body (peripheral resistance increase).

As used herein, the term “heart failure” is used in the broadest sense employed in the subject art. The term refers to the syndrome which is caused by disorders of cardiac function that pushes out sufficient blood flow. The term includes decline of cardiac output, accompany with an increase of the venous pressure, and the resultant various clinical symptoms. For example, a cause of heart failure of the present invention may include valvular heart disease, ischemic heart diseases, congenital heart diseases, dilated cardiomyopathy, hypertrophic cardiomyopathy, atrial septal defect, ventricular septal defect and symptomatic heart disease. Specifically, the valvular heart disease of the present invention includes aortic regurgitation, aortic stenosis, mitral regurgitation and mitral stenosis. Unlike atrial fibrillation, the reduction of cardiac outputs is the cause of heart failures. Namely, in the case of atrial fibrillation, for example, lone atrial fibrillations, blood circulation becomes dysfunctional because parts of the heart lack of coordination and contract frequently and finely and thus cardiac outputs are inconsistent.

As used herein, the term “marker” refers to a characteristic biological factor which shows genetic or expressive nature of a living organism. Typically, the marker may be a nucleic acid and RNA which is a gene, or gene product such as protein. Such markers can be measured by any appropriate biological assay known in the art. Furthermore, existence of markers can be used as an indicator of disease state or condition.

As used herein, the term “immunoassay method” refers to any assay method using immunological technology, such as antigen-antibody reaction. The term may include, for example, dot blot assays, western blot, enzyme-linked immunosorbent assay (EIA), a solid-phase enzyme immunoassay (ELISA), radioimmunoassay (RIA), an electro chemiluminescence immunoassay (ECLIA), chemiluminescent immunoassays (CLIA), chemiluminescent enzyme-linked immunosorbent assay (CLEIA), and competitive protein binding assay.

As used herein, the term “diagnosis” refers to identifying various parameters relevant to disease, disorder and condition of a subject and determining current state or future of such disease, disorder or condition. By employing the method, device and system of the present invention, conditions in the body can be examined. Obtained information may be used to select disease, disorder or condition in the subject and formulations or methods for treatment or prevention to be administered, and the like. As used herein, the term “diagnosis” refers to diagnosing current conditions in a restricted sense. However, it includes “prior diagnosing” in a broad sense.

In the present invention, the term “IR-” is often used as a form attached to other terms. As used herein, the term “IR-” refers to immunoreactive. “Immunoreactive” refers to “having reactivity” with antibody for detection.

In the present invention, “measurement” of pro-BNP108 and the like may be carried out by any method known in the art. Namely, it can be carried out by a known method for measuring protein, for example, immunological techniques employing a polyclonal antibody or monoclonal antibody specifically directed to a target protein (for example, proBNP-108 and the like), such as Western blotting methods, EIA method, RIA method, FIA method, chemistry luminescence immunoassay method or ECLIA method. Assay employing an antibody can refer to Antibodies: A Laboratory Manual (Harlow and Lane, Cold Spring Harbor Laboratory, 1988), Japanese Laid-Open Publication No. 10-160735 and the like.

In the present invention, separation between proBNP-108 and BNP-32 can be carried out by employing elution point, for example, in gel filtration high-performance liquid chromatography (HPLC) (for example, TSK gel G2000SWXL column (TOSOH), etc.). If such a gel filtration technique is employed, separation of proBNP-108 from BNP-32 can be performed. This separation can be attained by applying sample to gel filtration and comparing with elution point of standard. Moreover, glycosylated proBNP-108 and non-glycosylated proBNP-108 (for example, recombinant type) can also be distinguished by setting up conditions more strictly. However, for purpose of this invention, it is not necessarily required to distinguish glycosylated proBNP-108 from non-glycosylated proBNP-108 (for example, recombinant form).

In the diagnostic methods of the present invention, preferably, in order to measure proBNP-108 with the above-mentioned assay, the antibody which can recognize and bind to specific amino acid sequence of proBNP-108 but not to BNP-32 is used (available from, for example, BIO-RAD, Phoenix, Hytest and the like). Antibodies employed in the present invention may be any of polyclonal antibodies or monoclonal antibodies that can recognize the above-mentioned proteins. Antibodies used in the present invention can be manufactured according to known methods for producing antibody or antiserum, using target protein as an antigen.

To prepare cells producing monoclonal antibody, a protein or the like targeted in the present invention may be administered to a site of an animal which can produce antibody alone or with carrier or diluents. To enhance the capability of antibody production, complete Freund's adjuvant or incomplete Freund's adjuvant may be used in time of administration. Administration is usually performed once every two to six weeks and about total of 2 to 10 times. Animals used may include, for example, mammals such as monkey, rabbit, dog, guinea pig, mouse, rat, sheep, goat and the like. Preferably, mouse and rat are available. Measurement of antibody titers in antiserum can be performed, for example, by measuring activity of labeling agent bound to the antibody after reacting after-mentioned labeled protein or the like, described below, with antiserum.

An animal immunized with an antigen may be selected, for example, an individual mouse in which antibody titers has been raised, spleen or lymph nodes are removed 2-5 days after the final immunization and antibody-producing cells included therein may be fused with myeloma cells (for example, NS-1, P3U1, SP2/0, etc.) to prepare hybridoma producing monoclonal antibody. Fusing can be carried out according to a known method, for example, Koehler and Milstein's method (Nature 256: 495 (1975)). A fusion promoter may include, for example, polyethylene glycol (PEG), or Hemagglutinating Virus of Japan (HVJ). Preferably, PEG is employed.

Polyclonal antibodies can be prepared according to a known method or method similar thereto it. For example, such antibodies may be obtained according to the following step; preparing a complex of an immunogen (antigens such as proteins of the present invention) and carrier; immunizing against a mammal in similar manner to the above described methods for monoclonal antibodies; collecting a matter containing the antibody directed to the immunogen such as protein of the invention and the like from the mammal; separating and purifying of such antibodies.

### (Diagnostic Methods)

In one aspect, the present invention provides a method for determining the overload of either atrium or ventricle. The method includes the step(s) of measuring A) proBNP-108 (SEQ ID NO: 2) and/or B) levels of BNP-32 (SEQ ID NO:3) in a sample from a subject. In this regard, the overload of either ventricle or atrium can be determined, heart condition can be determined and heart condition such as heart failure can be determined more precisely. Such a utility of the method may include, for example, grasp of disease condition or progression of aortic stenosis, aortic regurgitation, mitral regurgitation, and atrial fibrillation.

In one embodiment, the measurement may determine proBNP-108/total BNP in the sample from the subject. The ratio of proBNP-108/total BNP can be calculated by dividing the amount of proBNP-108 by the amount of total BNP. Here, to measure total BNP, the amount of various molecular forms of BNP (for example, pre-pro-form, proBNP-108 (pro-form) and BNP-32 (mature form)) are measured respectively and these resultant values are put together. Alternatively, the measured result by employing monoclonal antibodies, which can recognize common region (for example, region of 77-108), can be substituted for the amount of total BNP.

In another aspect, the present invention provides a method for determining levels of progressing or treatment effect of heart failure. The method comprises the step of measuring levels of proBNP-108 in a sample from a subject. Alternatively, the method comprises a step of measuring the ratio of proBNP-108/total BNP in the sample from the subject.

As used herein, the term “progression of heart failure” refers to condition or stage of heart failure, or the transition thereof. Progression of heart failure can be classified according to the following criteria: symptom such as fatigability and breathlessness (NYHA cardiac function classification), the level of cardiac expansion and pulmonary congestion on chest radiography, expansion of left ventricular chamber by echocardiography, cardiac function, diameter of inferior vena cava, Doppler index, and the like.

The present inventors found that progression of heart failure and proBNP-108 levels have the following correlation: with progression of heart failure, proBNP-108 increases and the ratio of proBNP-108/total BNP also increases simultaneously. An increase in proBNP-108 levels allows prediction that symptom, cardiac expansion and pulmonary congestion on a chest radiography, expansion of left ventricular chamber by echocardiography, cardiac function, diameter of inferior vena cava, Doppler index, or the like has been excerbated. Therefore, the method of the present invention improves capability of diagnosis in comparison to the conventional diagnosis employing only measurement of BNP.

As used herein, the term “level of heart failure treatment” refers to a level which indicates how much heart failure has been improved by such a heart failure treatment, and can be used in evaluation of the treatment employed, or the determination of treatment plan thereafter. The level of heart failure can be determined according to the following criteria: symptom such as fatigability and breathlessness (NYHA cardiac function classification), cardiac expansion and pulmonary congestion on chest radiography, expansion of left ventricular chamber by echocardiography, cardiac function, diameter of inferior vena cava, Doppler index, and the like. Since proBNP-108 level is a biochemical marker which responds prior to these levels of heart failure treatment effect, the method of the present invention is superior to the conventional diagnosis employing only measurement of BNP. Using the present invention, staging of heart failure patient or its prognosis, determination of curative effect of beta blocking agent and ACE inhibitor for heart failure patient, grasp of dilated cardiomyopathy and determination of curative effect of dilated cardiomyopathy, grasp of myocardial infarction and grasp of remodeling after infarction and typing of hypertrophic cardiomyopathy can be conducted. The increase in proBNP-108 can be used as an index which indicates decline of right ventricular function. Moreover, the increase in proBNP-108 level or the ratio of proBNP-108/total BNP can be employed as indicative of pressure overload in disease with right ventricular overload.

The present invention also can be employed as indicative of treatment of heart failure. For example, it has been found in decompensated heart failures, that both BNP-32 level and proBNP-108 level increase. Moreover, when heart failures excerbate, these values and proBNP-108/total BNP increase. On the other hand, it has also been found that in the patient whose condition had improved in response to treatments, the levels of both BNP-32 and proBNP-108 decreased and proBNP-108/total BNP also decrease. These values decreased similarly in the cases of spontaneous recovery. Therefore, each of BNP-32, proBNP-108 and proBNP-108/total BNP can be used for determining level of improvement of heart failure, level of treatment or level of cure.

Treatments which may be determined in the present invention may include, for example, medicinal treatments and surgical treatments and the like. Medicinal treatments may include, for example, the treatment with ACE and the like. Surgical treatments may include aortic valve replacement (AVR), mitral valve replacement (MVR), double valve replacement (DVR), coronary-arteries bypass transplant (CABG), maze method, Dor method, and the like.

As used herein, “the overload of atrium” refers to overloading of atrium and may include, for example, heart failure with atrial overload.

As used herein, “the overload of ventricle” refers to overloading of ventricle and may include, for example, heart failure with ventricular overload.

It is known that heart failure is caused by overloading of either atrium or ventricle or both. It is also known that depending on its type, subsequent medical treatment and the like should be selected.

As used herein, “heart failure with atrial overload” refers to heart failure of the type where an atrium is mainly overloaded. Heart failure with atrial overload may include, for example, mitral stenosis, mitral regurgitation, atrial septal defect and the like. In the case of heart failure with atrial overload, it is preferable to administer a medicament which reduces atrial overload, which includes diuretics, vasodilators, hANP or the like.

As used herein, “heart failure with ventricular overload” refers to heart failure of the type where a ventricle is mainly overloaded. Heart failure with ventricular overload may include, for example, aortic regurgitation, aortic stenosis and the like can be recited, for example. In the case of heart failure with ventricular overload, it is preferable to administer a medicament which mainly acts on the ventricle, or implement appropriate treatment.

Such a medicament includes ACE inhibitor, angiotensin receptor antagonist, beta blocker, aldosterone antagonist, etc. can be recited. Moreover, the determination of the timing for treating an aortic valve replacement may be conducted

Regarding the ratio of proBNP-108/total BNP, generally in the case of atrial overload, the value thereof is smaller compared with the case of ventricular overload.

For example, when determination is made using fractions, which was obtained by applying plasma from a patient with heart failure to gel filtration HPLC, in the case of ventricular overload, the ratio of proBNP-108/total BNP is in about 0.3-0.9, preferably in about 0.4-0.8. In the case of atrial overload, the value is in about 0.1-0.5, preferably in about 0.2-0.4. The value may vary according to change of specific symptom of such disease, and may have tendency of dispersion. For example, in mitral regurgitation patients, value is about 0.15±0.03.

Moreover, when determination is made using pericardial effusions, the resultant value tends to be higher than the same of plasma. For example, the value is in 0.7-0.9.

Additionally, determination can also be attained by the amounts or concentrations of proBNP-108 as an indicator. In the case of heart failure with ventricular overload, proBNP-108 concentration may be in 0-2000 pg/mL, preferably 50-100 pg/mL and the like. In the case of heart failure with atrial overload, the concentration may be in 0-400 pg/mL.

In other embodiment, pericardial effusions, blood, or its processed product (for example, plasma) and the like can be used as a sample, but it is not limited thereto. It should be understood that criteria for determination may vary depending on the sample selected. Based on the description described herein, a skilled person can appropriately select and decide such criteria.

Such sample can be obtained from the patient who is suspected of at least one disease selected from the group which consists of heart failure, atrial fibrillation, mitral regurgitation, and aortic stenosis.

In all embodiments of the method of the present invention, other markers employed in the art may also be used. Employing the marker of the present invention in combination with such other markers is advantageous in that more variety of types of disease are identifiable in one diagnostic step.

### (Use of a Marker)

In one aspect, the present invention provides the use of proBNP-108 as a marker for determining the overload of either atrium or ventricle in a subject. Conventionally, although using BNP-32 as a marker for heart failure and the like is known, measuring proBNP-108 per se has not been performed. However, our invention discloses for the first time that proBNP-108 can be used as a marker for certain utility. The more detailed embodiment of use as a marker can be carried out according to the description in the paragraph of (Diagnostic methods).

In another aspect, the present invention provides the use of a combination of proBNP-108 and BNP-32 as a marker for determining the overload of either atrium or ventricle in a subject. By using both markers, the overloaded of atrium or ventricle can be detected. As a result, disease condition or progression of aortic stenosis, aortic regurgitation, mitral stenosis, mitral regurgitation and atrial fibrillation can also be grasped. Moreover, the more detailed embodiment of use as a marker can be carried out according to the description in the paragraph of (Diagnostic methods).

In another aspect, the present invention provides the use of proBNP-108 as a marker for determining the level of progression of heart failure, or heart failure medical treatment. In this case, a similar purpose of a detection of proBNP-108, staging of heart failure patient or its prognosis, determination of curative effect of beta blocking agent and ACE inhibitor for heart failure patient, grasp of dilated cardiomyopathy and determination of curative effect of dilated cardiomyopathy, grasp of myocardial infarction and grasp of remodeling after infarction and typing of hypertrophic cardiomyopathy can also be conducted. The increase in proBNP-108 can be used as an index which indicates decline of right ventricular function in disease with right ventricular overload. Moreover, the increase in proBNP-108 can be employed as an indicator of showing the pressure overload in disease with right ventricular overload. Moreover, the more detailed embodiment of use as a marker can be carried out according to the description in the paragraph of (Diagnostic methods).

### (Diagnostic Kit)

In one aspect, the present invention provides a diagnostic kit for determining the overload of atrium or ventricle in a subject comprising a substance that specifically binds to proBNP-108.

In another aspect, the present invention provides a diagnostic kit for determining the overload of atrium or ventricle in a subject comprising a substance that specifically binds to proBNP-108, and a substance that specifically binds to BNP-32. The more detailed embodiment of such diagnostic kit can be carried out according to the description in the paragraph of (Diagnostic methods).

In another aspect, the present invention provides a diagnostic kit for determining the overload of which atrium or ventricle in a subject comprising a substance that specifically binds to proBNP-108. The more detailed embodiment of such diagnostic kit can be carried out according to the description in the paragraph of (Diagnostic methods).

As used herein, the term “a substance that specifically binds to proBNP-108” may be any substance as long as proBNP-108 can be measured. For example, such a term includes, but is not limited to, an antibody that specifically binds to proBNP-108. More detailed embodiment of an antibody which specifically binds to proBNP-108 can be carried out according to the description in the paragraph of (Diagnostic methods). Such a substance may be any substance or even other element as long as such a substance has specificity and that the intended purpose can be attained. Such a substance includes, but is not limited to, for example, protein, polypeptide, oligopeptide, peptides, polynucleotide, oligonucleotide, nucleotide, and nucleic acids (for example, cDNA, DNA like genomic DNA, and RNA like mRNA), polysaccharide, oligosaccharide, lipid, organic molecules (for example, hormone, ligand, messenger, organic molecule, molecule synthesized by combinatorial chemistry technology, and low molecules which may be used as drug (for example, low molecular ligand etc.)), and complexed molecule thereof.

As used herein, the term “a substance that specifically binds to BNP-32” may be any substance as long as BNP-32 can be measured. Such a substance may include, but is not limited to, for example, an antibody specifically binds to BNP-32. More detailed embodiment of an antibody that specifically binds to BNP-32 can be carried out according to the description in the paragraph of (Diagnostic methods).

As used herein, the term “specifically(ally)” refers to an affinity over a specific biological factor that is equivalent or higher than the affinity over the non-relevant polypeptide (especially, identity thereof is less than 30%). Preferably, it means significantly higher (for example, statistically significant). Such the affinity can be measured by binding assay, for example.

As used herein, the term “bond” or “bind(ing) (to)” refers to the physical interaction or the chemical interaction between two proteins or compounds, related proteins and compounds or the combination thereof. Bonds include ionic bond, non-ionic bond, hydrogen bond, van der Waals bond, hydrophobic interaction and the like. The physical interactions (binding) may be direct or indirect. Indirect binding is mediated or caused by effect of other proteins or compounds. Direct binding refers to interaction, not being mediated or caused by the effect of other proteins or compounds, and not accompanying with other substantial chemical intermediates.

### (General Techniques)

Molecular biological techniques, biochemical techniques, and microorganism techniques as used herein are well known in the art and commonly used, and are described in, for example, Sambrook J. et al. (1989). Molecular Cloning: A Laboratory Manual, Cold Spring Harbor and its 3rd Ed. (2001); Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirely) of each of these publications are herein incorporated by reference.

Reference including scientific literature, patents, published patent applications and publications cited herein is incorporated by reference as if set forth fully herein.

The preferred embodiments of the present invention have been heretofore described for better understanding of the present invention. Hereinafter, the present invention will be described by way of examples. Examples described below are provided only for illustrative purpose. Accordingly, the scope of the present invention is not limited by the embodiments and examples specified herein except as by the appended claims.

### EXAMPLES

Hereinafter, the present invention is exemplified, but not restricted by the following Examples. Unless otherwise specified, reagents used herein were commercially available. Moreover, in regard of a patient, study was performed after obtaining patient's informed consent and fulfilling any certified international ethical standards.

Patients' Study

A total of 132 Japanese patients with heart failure (65 men and 67 women; age range, 25 to 90 years, mean age, 67±11 years) were enrolled in this study. The primary cause of heart failure was valvular heart disease (n=55), ischemic heart disease (n=49), congenital heart disease (n=13), dilated cardiomyopathy (n=8), hypertrophic cardiomyopathy (n=8) and others (n=9). Patients with symptomatic heart failure were receiving agents, including angiotensin-converting-enzyme inhibitors/angiotensin-receptor blocker (67%), digitalis (35%) and diuretics (72%) and the like. New York Heart Association (NYHA) functional class was as follows: class I, n=31, mean age 65±10 years, 12 men and 19 women; class II, n=69, 68±12 years, 38 men and 31 women; class III, n=24, 71±8 years, 12 men and 12 women; and class IV, n=8, 63±11 years, 3 men and 5 women.

Informed consent was obtained from each patient, and the protocol was approved by the ethics committee of our institute and/or was carried out according to the recommendation of the ethical committee of Dokkyo Medical University.

### Example 1

### Comparison of Control Subjects, Patients with Atrial Fibrillation, and Patients with Heart Failure in Plasma BNP-32 Levels and proBNP-108 Levels)

We measured plasma BNP-32 and proBNP-108 concentrations in control subjects, patients with atrial fibrillation, and patients with heart failure. The patients with atrial fibrillation had lone atrial fibrillation; other cardiovascular diseases were excluded by physical examination, clinical test, chest radiography, electrocardiography and echocardiography. The controls were 10 subjects (4 men and 6 women; age range 35 to 77 years; means age, 65±12 years) with normal findings on physical examination, clinical test, chest radiography, electrocardiography and echocardiography. The primary cause of heart failure was evaluated on the basis of the medical history, physical examination, chest radiography, electrocardiography, echocardiography and/or cardiac catheterization. Patients' characteristics are presented in Table 1 below.

TABLE 1
Clinical Characteristics of Heart Failure Patients

| | | Atrial | Heart |
|---|---|---|---|
| | Control | fibrillation | failure |
| Number | 10 | 18 | 132 |
| Sex(male/female) | 4/6 | 12/6 | 65/67 |
| Age (yrs) | 65 ± 12 | 68 ± 9 | 67 ± 11 |
| Etiology | | | |
| AR or AS | | | 30 |
| MR or MS | | | 25 |
| IHD | | | 49 |
| DCM | | | 8 |
| HCM | | | 8 |
| ASD | | | 7 |
| VSD | | | 6 |
| other | | | 9 |
| NYHA I | | | 31 |
| II | | | 69 |
| III | | | 24 |
| IV | | | 8 |
| Total IR-BNP (pg/mL) | 20 ± 7 | 126 ± 71* | 430 ± 764*† |
| IR-BNP-32 | 9 ± 3 | 68 ± 37* | 256 ± 406*† |
| IR-proBNP-108 | 12 ± 5 | 58 ± 31* | 174 ± 369*† |

Values are mean ± S.D., AR: aortic regurgitation, AS: aortic stenosis, MR: mitral regurgitation, MS: mitral stenosis, IHD: ischemic heart disease, DCM: dilated cardiomyopathy, HCM: hypertrophic cardiomyopathy, ASD: atrial septal defect, VSD: ventricular septal defect, NYHA: New York Heart Association, BNP: brain ntriuertc peptide
*p < 0.05 vs. Control,
†p < 0.05 vs. Atrial fibrillation,

### Blood Sampling

Blood samples (3 mL) were withdrawn from all subjects via the antecubital vein. Blood was immediately transferred into chilled glass tube containing disodium EDTA (1 mg/mL) and aprotinin (500 U/mL). Blood was centrifuged immediately at 4° C. and the plasma was frozen and stored at −80° C. until measurement.

Measurement for Plasma BNP-32 and proBNP-108

Plasma or pericardial fluid samples which had been stored at −80° C. were extracted with Sep-Pak C18 cartridges (Waters, Milford, Mass., USA). The cartridges were prewashed sequentially with 5 mL each of chloroform, methanol, 50% acetonitrile containing 0.1% trifluoroacetic acid (TFA), 0.1% TFA, and saline as reported previously (Biochem. Biophys. Res. Commun. 1992; 185: 760-7). Plasma (3 mL) was acidified with 3 mL of saline containing 28 μL of 1 mol/L HCl and diluted with 3 mL of saline and then loaded onto a Sep-Pak C18 cartridge. After washing with 5 mL each of saline, 0.1% TFA, and 20% acetonitrile containing 0.1% TFA, the absorbed materials were eluted with 4 mL of 50% acetonitrile containing 0.1% TFA. The elute was lyophilized. The lyophilized material was then dissolved in 30% acetonitrile containing 0.1% TFA and subjected to gel filtration high performance liquid chromatography (HPLC) on a TSK gel G2000SWXL column (7.8×300 mm, Tosoh, Tokyo, Japan) in the same buffer at a flow rate of 0.2 mL/min. Column effluents were fractionated from 20 min after the injection into polypropylene tubes containing bovine serum albumin (50 μg) and Triton X-100 (25 μg). Each fraction was lyophilized and dissolved in radioimmunoassay buffer, centrifuged and the clear solution was then submitted to fluorescent immunoenzyme assay for BNP-32 (Tosoh). Details of the fluorescent immunoenzyme assay for BNP-32 were reported previously, and monoclonal antibodies for BNP-32 from Shionogi (Osaka, Japan) were used in this assay system (Iryo to Kensakiki-Siyaku. 2005; 28(3): 255-261).

ProBNP-108/total BNP ratio was calculated based on the summation of a high molecular weight (MW) immunoreactivity (proBNP-108) and a low MW immunoreactivity (BNP-32) with the following formula:

proBNP-108/total BNP ratio=proBNP-108/(proBNP-108+BNP-32)

Since very recent studies have revealed that glycosylated pro-BNP of MW about 35K is circulating in plasma (J. Am. Coll. Cardiol. 2007; 49: 1071-8 and Biochem. Biophys. 2006; 451: 160-6), we purchased recombinant proBNP-108 and glycosylated proBNP-108 (HyTest, Finland), and synthetic BNP-32 (Peptide Institute, Japan), and examined the elution positions of these three peptides in the gel filtration. To evaluate the cross-reactivity of proBNP-108 and glycosylated proBNP-108 in the immunoenzyme assay, each peptide was desalted with a Monotip C18 cartridge (GL Sciences, Tokyo, Japan) by the manufacturer's protocol. An aliquot of the desalted peptide was subjected to the immunoenzyme assay and another aliquot was submitted to amino acid analysis (L-8500 analyzer, Hitachi, Tokyo, Japan) after acid hydrolysis at 110° C. for 22 hours to estimate content of each peptide.

### Statistical Analysis

All values (variations) are expressed as mean±SD. The statistical significance of differences between 2 groups was evaluated with Fisher's exact test or unpaired Student's t test, as appropriate. Log transformation was used to normalize the distribution of plasma peptide levels, if appropriate. Categorical variables were compared with the use of the chi-square test. Variables were compared among 3 groups by means of 1-way analysis of variance followed by Boneferoni's multiple comparison tests. Correlation coefficients were calculated by linear regression analysis. P values of <0.05 were considered to indicate statistical significance.

### Results

1) Measurements of Immunoreactive BNP-32 and Immunoreactive proBNP-108 Concentrations.

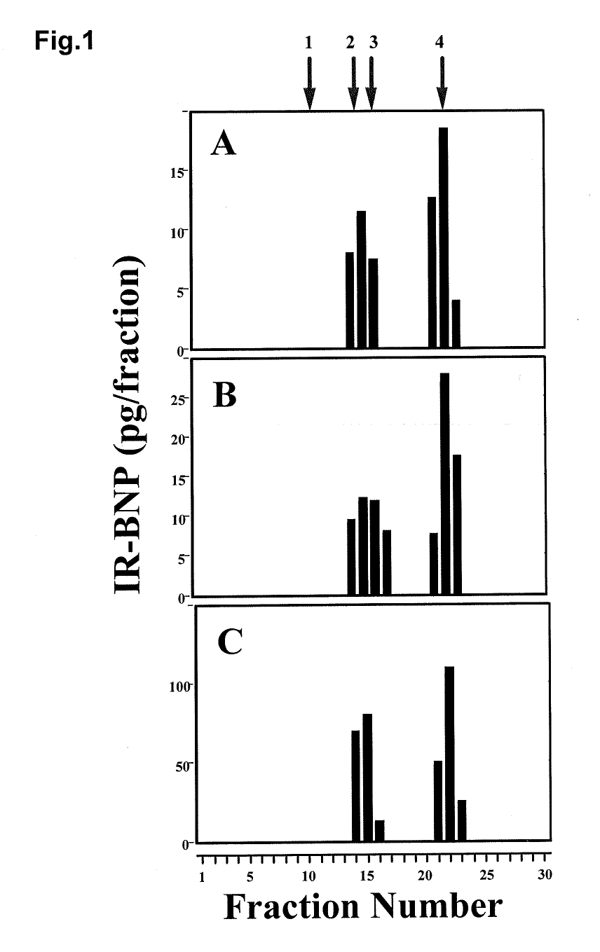

To characterize the molecular forms of BNP in human plasma, a peptide fraction obtained by reverse phase C-18 column condensation was subjected to gel filtration HPLC on a TSK gel G2000SWXL column. As shown in FIG. 1, two peaks of IR-BNP, IR-proBNP-108 (high molecular weight immunoreactive (IR-) BNP including both proBNP-108 and glycosyl proBNP-108) and IR-BNP-32 (low molecular weight IR-BNP mainly consisting of BNP-32), were constantly observed in control and the patients with atrial fibrillation and heart failure. The first peak (IR-proBNP-108) was observed in fractions #9-16 of MW larger than 13K, and the second peak (IR-BNP-32) was in fractions #18-21 of MW 3.5K corresponding to BNP-32. In this gel filtration HPLC, recombinant proBNP-108 and glycosylated proBNP-108 were each eluted mainly in fraction #14 and fraction #15, and not separable with each other.

Cross-reactivity of proBNP-108 and glycosylated proBNP-108 in the fluorescent immunoenzyme assay for BNP-32 was estimated to be 52.8% and 72.0% based on their net contents determined by amino acid analysis. Both pro-BNP-108 and glycosylated proBNP-108 share the C-terminal BNP-structure and the fluorescent immunoenzyme assay for BNP-32 strictly recognizes the BNP-32 structure. Although there was a possibility that carbohydrate chains of the N-terminal proBNP-76 may interfere the antibody recognition of glycosylated proBNP-108, this possibility was denied by higher cross-reactivity observed for glycosylated proBNP-108. Rather the difference in the cross-reactivity is deduced to be derived from differences in the structural fidelity of BNP-32 moiety of non-glycosylated and glycosylated proBNP-108s, since these two proBNP-108s were produced by the different recombinant technology from E. coli and HEK293 cells. Thus, complete molecules of the glycosylated and non-glycosylated proBNP-108s are expected to show the cross-reactivity more than 70% and we did not correct the concentration of IR-proBNP-108 based on the cross-reactivity. On the other hand, synthetic BNP-32 was constantly measured in a range of 100-110% in this assay.

2) Plasma Concentrations of IR-BNP-32 and IR-proBNP-108 in Control, Atrial Fibrillation, and Heart Failure.

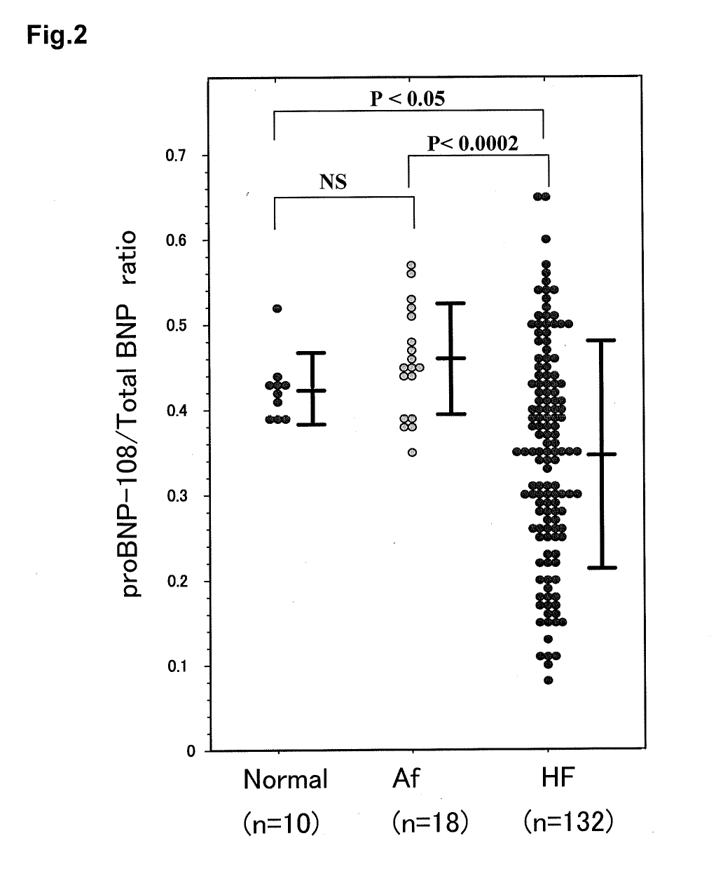

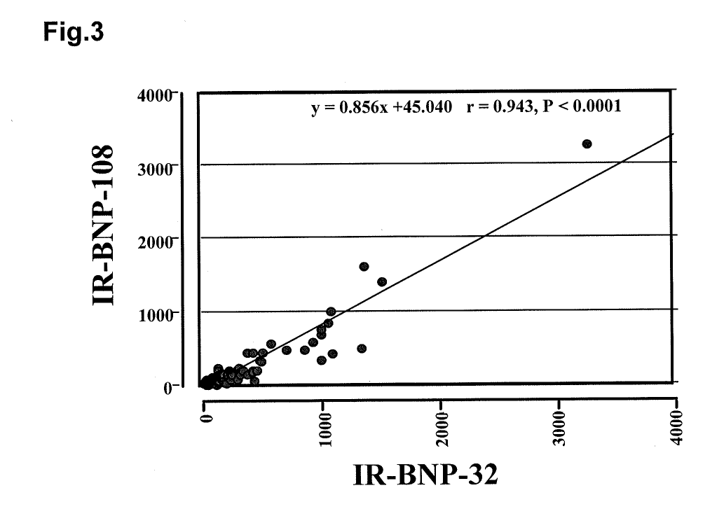

The proBNP-108/total BNP ratio is shown in FIG. 2. Since recombinant proBNP-108 and glycosylated proBNP-108 could not be separated from each other, proBNP-108/total BNP ratio is calculated based on the formula: proBNP-108/total BNP ratio=proBNP-108/(prBNP-108+BNP-32). The proBNP-108/total BNP ratio was narrowly distributed in control and atrial fibrillation; however, the proBNP-108/total BNP ratio was widely distributed in patients with heart failure. As a result, the mean proBNP-108/total BNP ratio in heart failure was significantly lower in heart failure than in control or atrial fibrillation. The mean concentrations of IR-BNP-32 and IR-proBNP-108 are shown in Table 1. Not only IR-BNP-32, but also IR-proBNP-108 was significantly higher in heart failure than the other two groups. There was a good correlation between IR-BNP-32 and IR-proBNP-108 levels measured by the present procedures (FIG. 3).

### Example 2

### Plasma proBNP-108/Total BNP Ratio in Patients with Heart Failure: Comparison Between Heart Failure with Atrial Overload and Ventricular Overload

We divided 62 of the 132 patients with heart failure in Example 1 into two groups: heart failure with atrial overload and heart failure with ventricular overload. Heart failure with atrial overload included mitral stenosis, mitral regurgitation and atrial septal defect (n=32), whereas heart failure with ventricular overload included aortic regurgitation and aortic stenosis (n=30). We compared the ratio of proBNP-108/(BNP-32+proBNP-108) in heart failure with atrial overload to that in heart failure with ventricular overload. The patients' characteristics are presented in Table 2.

TABLE 2
Clinical Characteristics of Heart Failure Patients with Ventricular Overload and Atrial Overload

| | | | | Total IR-BNP | IR-BNP-32 | IR-BNP-108 | proBNP-108/Total |
|---|---|---|---|---|---|---|---|
| Diagnosis | Number | Age (yrs) | (male/female) | (pg/mL) | (pg/mL) | (pg/mL) | BNP ratio |
| Ventricular | 30 | 68 ± 9 | (18/12)* | 442 ± 517* | 256 ± 282* | 186 ± 236* | 0.40 ± 0.10* |
| Overload | | | | | | | |
| AR | 17 | 63 ± 10 | (11/6) | 522 ± 657 | 304 ± 362 | 218 ± 298 | 0.39 ± 0.09 |
| AS | 13 | 71 ± 6 | (7/6) | 338 ± 223 | 193 ± 122 | 144 ± 113 | 0.41 ± 0.11 |
| Atrial | 32 | 66 ± 9 | (4/28) | 176 ± 224 | 121 ± 141 | 55 ± 92 | 0.27 ± 0.13 |
| Overload | | | | | | | |
| MR | 19 | 69 ± 7 | (3/16) | 238 ± 270 | 157 ± 171 | 81 ± 110 | 0.32 ± 0.14 |
| MS | 6 | 62 ± 5 | (0/6) | 87 ± 23 | 74 ± 19 | 13 ± 5 | 0.15 ± 0.03 |
| ASD | 7 | 64 ± 13 | (1/6) | 146 ± 202 | 107 ± 140 | 39 ± 62 | 0.23 ± 0.06 |

Values are mean ± S.D., AR: aortic regurgitation, AS: aortic stenosis, MR: mitral regurgitation, MS: mitral stenosis,
*p < 0.05 vs. atrial overload

### Blood Sampling

Blood samples (3 mL) were withdrawn from all subjects via the antecubital vein. Blood was immediately transferred into chilled glass tube containing disodium EDTA (1 mg/mL) and aprotinin (500 U/mL). Blood was centrifuged immediately at 4° C. and the plasma was frozen and stored at −80° C. until measurement.

Measurements of BNP-32 and proBNP-108 concentrations To calculate the proBNP-108/total BNP ratio, both plasma BNP-32 and plasma pro-BNP-108 concentration were measured in similar manner to Example 1.

### Statistical Analysis

All values (variations) are expressed as mean±SD. The statistical significance of differences between 2 groups was evaluated with Fisher's exact test or unpaired Student's t test, as appropriate. Log transformation was used to normalize the distribution of plasma peptide levels, if appropriate. Categorical variables were compared with the use of the chi-square test. Variables were compared among 3 groups by means of 1-way analysis of variance followed by Boneferoni's multiple comparison tests. Correlation coefficients were calculated by linear regression analysis. P values of <0.05 were considered to indicate statistical significance.

### Results

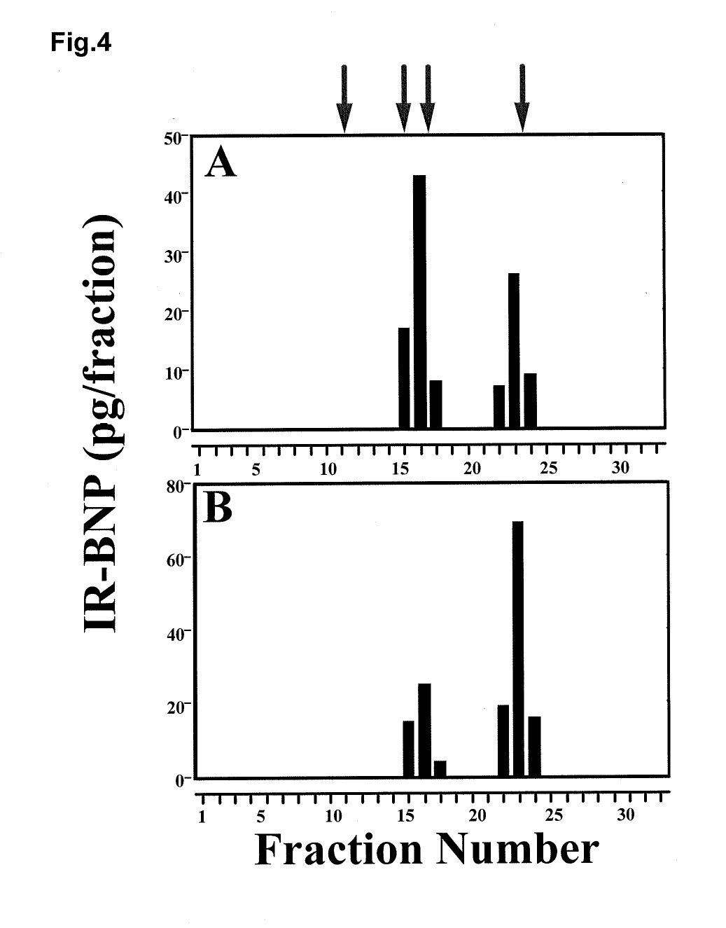

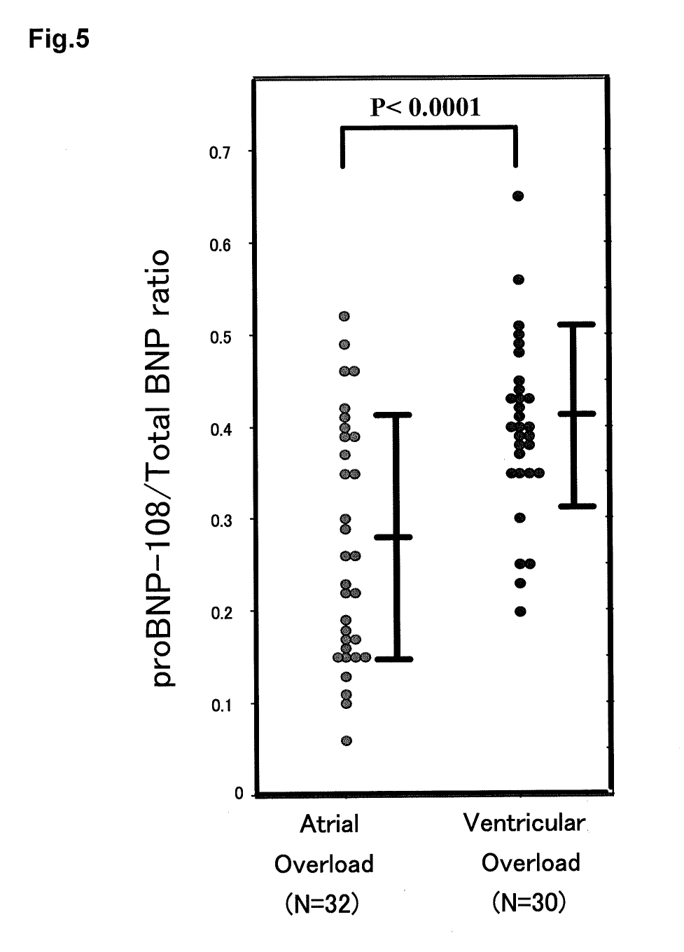

To investigate the reasons for the wide distribution of the proBNP-108/total BNP ratio, we measured proBNP-108/total BNP ratio in heart failure patients with atrial overload and those with ventricular overload. As shown in FIG. 4, two peaks of IR-BNP were observed in both groups. Interestingly, IR-BNP-32 peak was more dominant than IR-proBNP-108 peak in heart failure with atrial overload (FIG. 4-B); in contrast, IR-proBNP-108 and IR-BNP-32 peaks were nearly equivalent in heart failure with ventricular overload (FIG. 4-A). As a result, the mean proBNP-108/total BNP ratio was higher in heart failure with ventricular overload than that in heart failure with atrial overload (FIG. 5).

### Discussion

In the present study, we measured plasma IR-BNP-32 and IR-proBNP-108 levels in heart failure with atrial overload and ventricular overload and compared proBNP-108/total BNP ratios in these two conditions. We found that proBNP-108/total BNP ratio was higher in heart failure with ventricular overload than in heart failure with atrial overload, although plasma levels of both peptides correlated with each other.

### Example 3

### Measuring Concentrations of IR-BNP-32 and IR-proBNP-108 in Ventricular and Atrial Tissue

In order to elucidate mechanism in which proBNP-108/total BNP ratio of heart failure with atrial overload is lower than that of heart failure with ventricle overload, we analyzed molecular forms of BNP in atrial and ventricular tissues in other patients who underwent cardiac surgery (n=11). Atrial tissues were obtained from patients with mitral disease (n=6) and autopsy case (n=1), and ventricular tissue samples were obtained by cardiac operation (n=5), and autopsy case (n=1). The characteristics of the patients who offered atrial tissue and ventricular tissue samples are presented in Table 3.

TABLE 3
Clinical Characteristics of Heart Failure Patients who
offered Left Atrial tissue and Left Ventricular tissue

| | Atrial | Ventricular |
|---|---|---|
| | tissue | tissue |
| Variables | | |
| Number | 7 | 6 |
| Age (yrs) | 56 ± 12 | 68 ± 13 |
| Sex (female/male) | 5/2 | 5/1 |
| Operation | | |
| MVR + maze | 5 | 0 |
| DVR + maze | 1 | 0 |
| Dor + CABG | 0 | 5 |
| Autopsy | 1 | 1 |
| Tissue total IR-BNP levels | 113 ± 130 | 17 ± 15 |
| (pg/mg tissue) | | |

Values are mean ± S.D., MVR; mitral valve replacement, DVR; dual valve replacement, CABG: coronary artery bypass graft

Sampling of Left Atrial Appendage Tissue and Left Ventricular Tissue

Resected samples of left atrial tissues were frozen from 7 patients (MVR+maze, n=5; DVR+maze, n=1; autopsy, n=1), frozen in liquid nitrogen, and stored at −80° C. Resected samples of left ventricular tissues were also obtained from 6 patients (coronary artery bypass grafting+Dor procedure, n=5; autopsy, n=1) frozen in liquid nitrogen, and stored at −80° C. Our goal was to obtain biochemical evidence regarding the molecular forms of BNP in the atrial and ventricular tissue.

Assay for Cardiac Tissue BNP-32 and ProBNP-108

The atrial and ventricular tissues obtained at cardiac surgery and autopsy were stored at −80° C., weighed, and boiled in 10 volumes of 1 mol/L acetic acid as described previously (J. Am. Coll. Cardiol. 2002; 39:288-94). Then, the tissues were homogenized with a Polytron mixer. The homogenate was centrifuged at 3,000×g, and the supernatant was centrifuged again at 15,000×g for 15 min. The second supernatant was extracted using a Sep-Pak C18 cartridge as described above in Example 1 for plasma. The eluate was lyophilized, and then subjected to gel filtration HPLC on a TSK gel G2000SWXL column. An aliquot of each fraction was lyophilized and IR-BNP was measured in a manner similar to Example 1.

### Statistical Analysis

All values (variations) are expressed as mean±SD. The statistical significance of differences between 2 groups was evaluated with Fisher's exact test or unpaired Student's t test, as appropriate. Log transformation was used to normalize the distribution of plasma peptide levels, if appropriate. Categorical variables were compared with the use of the chi-square test. Variables were compared among 3 groups by means of 1-way analysis of variance followed by Boneferoni's multiple comparison tests. Correlation coefficients were calculated by linear regression analysis. P values of <0.05 were considered to indicate statistical significance.

### Results

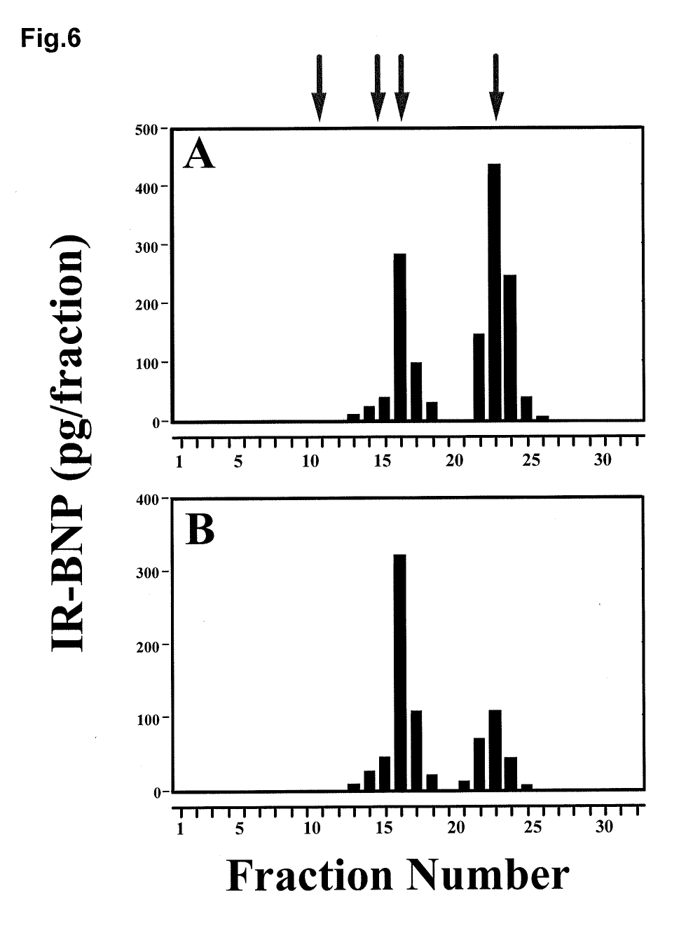

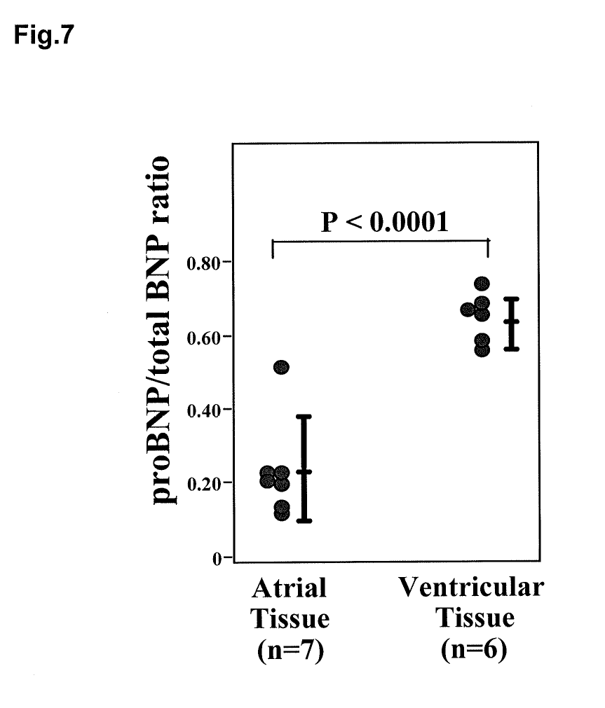

Similarly to the plasma samples, two IR-BNP peaks corresponding to BNP-32 and proBNP-108 were also observed in atrial and ventricular tissue. Interestingly, in atrial tissue, low MW IR-BNP peak corresponding to BNP-32 was the dominant molecular form as compared with high MW IR-BNP peak (n=7, 77±5%) (FIG. 6-A); in contrast, the high MW IR-BNP peak, corresponding to proBNP-108 was the dominant molecular form of IR-BNP in ventricular tissue (n=6, 66±4%, P<0.0001) (FIG. 6-B). Consequently, the mean proBNP-108/total BNP ratio was higher in ventricular tissue than in atrial tissue (FIG. 7).

To elucidate the reason for higher proBNP-108/total BNP ratio in heart failure with Ventricular overload than that in heart failure with atrial overload, we measured IR-BNP-32 and IR-proBNP-108 concentrations in atrial and ventricular tissue. Interestingly, more than 70% of IR-BNP was present as proBNP-108 in the ventricular tissue, whereas more than 75% of IR-BNP was present as BNP-32 in the atrial tissue.

Hino et al. (Biochem. Biophys. Res. Commun. 1990; 167: 693-700) previously demonstrated that two molecular forms of IR-BNP of MW 4K and MW 13-15K were present in human atrial extracts, which were identified to be BNP-32 and proBNP-108 by direct N-terminal sequencing of each peptide isolated using anti-BNP IgG immunoaffinity chromatography and reversed phase HPLC. They also reported that BNP-32 is the major molecular form in human atrial tissue, consistent with our results. However, to our best of knowledge, no study has previously examined the molecular form of BNP in human ventricular tissue. Goetze et al. (Eur. Heart J. 2006; 27: 1648-50) suggested the importance of atrium-derived BNP in heart failure, although it has received less attention.

Taken together, available evidence suggests that proBNP-108 rather than BNP-32 is the main molecular form of BNP in ventricular tissue and that the most of proBNP-108 is secreted from ventricle without processing. A recent report has shown that plasma level of proBNP-108 as well as that of BNP-32 is increased in patients with heart failure and that plasma proBNP-108 concentrations strongly correlate with plasma BNP-32 concentrations (J. Am. Coll. Cardiol. 2007; 49: 1193-202; J. Am. Coll. Cardiol. 2008; 51: 1874-82; Clin. Chem. 2007; 53: 866-73; and Clin. Chim. Acta 2003; 334: 233-9). Both of these findings are in agreement with our results.

### Example 4

### Measuring Concentrations of IR-BNP-32 and IR-proBNP-108 in Pericardial Fluid and Plasma in Patients with Heart Failure who Underwent Cardiac Surgery

To confirm the molecular form of BNP produced and secreted from the ventricular tissue, we studied 8 patients with heart failure who underwent cardiac surgery (aortic valve replacement, n=4; and mitral valve replacement, n=4). We measured proBNP-108 and BNP-32 levels in pericardial fluid and plasma in the patients. The characteristics of the patients who offered pericardial fluid are presented in Table 4.

TABLE 4
Clinical Characteristics of Heart Failure
Patients who Offered Pericardial Fluid

| | Heart Failure Patients |
|---|---|
| Variables | |
| Number | 8 |
| Age (yrs) | 66 ± 12 |
| Sex (female/male) | (4/4) |
| Plasma total IR-BNP-32 | 250 ± 123 |
| Operation | |
| MVR + maze | 2 |
| MVR | 2 |
| AVR | 4 |

Values are mean ± S.D., MVR: mitral valve replacement, AVR: aortic valve replacement,

Sampling of Plasma and Pericardial Fluid

Immediately after the incision of the pericardium, undiluted samples of pericardial fluid were obtained as reported previously (Clin. Sci. (London) 2002; 102: 669-77; and J Card Fail. 2004; 10: 321-7). At the same time, blood was withdrawn from the cannulated brachial artery. Samples were stored at −80° C. as described above.

### Measuring Concentrations of IR-BNP-32 and IR-proBNP-108 in Pericardial Fluid and Plasma

Plasma samples or pericardial fluid samples, which were stored at −80° C. were treated in similar manner to the method described in Example 1, measuring the levels of BNP-32 and proBNP-108 and calculating the proBNP-108/total BNP ratio.

### Statistical Analysis

All values (variations) are expressed as mean±SD. The statistical significance of differences between 2 groups was evaluated with Fisher's exact test or unpaired Student's t test, as appropriate. Log transformation was used to normalize the distribution of plasma peptide levels, if appropriate. Categorical variables were compared with the use of the chi-square test. Variables were compared among 3 groups by means of 1-way analysis of variance followed by Boneferoni's multiple comparison test. Correlation coefficients were calculated by linear regression analysis. P values of <0.05 were considered to indicate statistical significance.

### Results

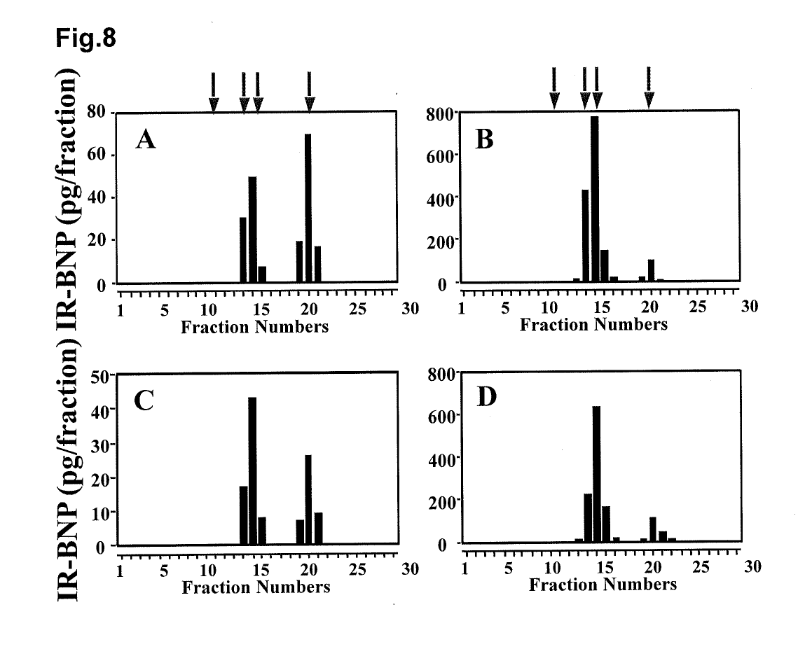

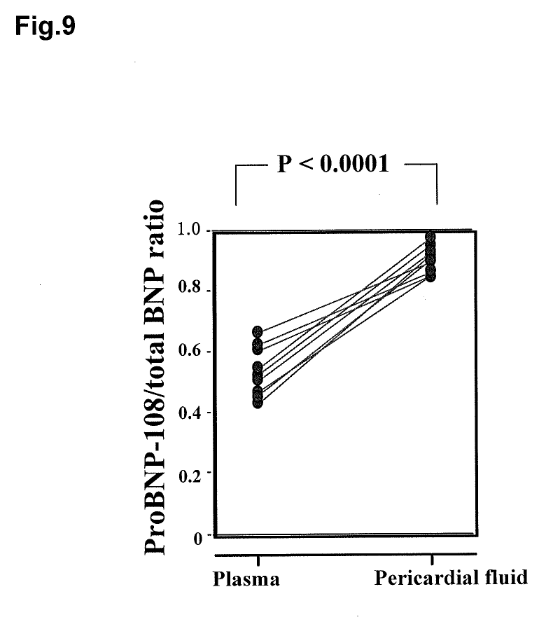

To confirm the molecular form of BNP produced and secreted from the ventricular tissue, we measured IR-BNP-32 and IR-proBNP-108 in pericardial fluid and plasma in 8 patients who underwent cardiac surgery. FIG. 8 shows the presence of two IR-BNP peaks corresponding to BNP-32 and proBNP-108 in plasma and pericardial fluid in both patients with mitral regurgitation (FIG. 8-A, B) and with aortic stenosis (FIG. 8-C, D). In plasma, IR-BNP-32 was the dominant molecular form in patients with mitral regurgitation, while IR-proBNP-108 was the dominant molecular form in patients with aortic stenosis; however, IR-proBNP-108 was exclusively the dominant molecular form in pericardial fluid in both types of patients. Consequently, the mean proBNP-108/total BNP ratio was greater in pericardial fluid than in plasma (FIG. 9).

Pericardial fluid is known to contain abundant levels of various bioactive substances produced in the heart (J. Am. Coll. Cardiol. 2007; 49: 1071-8; and Biochem. Biophys. 2006; 451: 160-6) and its composition is known to be similar to the interstitial fluid in the ventricle (Circulation 1996; 94: 610-3). In addition, the concentrations of bioactive substances, such as adrenomedullin, BNP, ANP, basic fibroblast growth factor, and vascular endothelial growth factor have been reported to be higher in pericardial fluid than in plasma (Clin. Sci. (Lond.) 2002; 102: 669-77; J. Card. Fail. 2004; 10: 321-7; Circulation 1996; 94: 610-3; and Heart 2002; 87: 242-6). Interestingly, our study indicates that most IR-BNP is present as IR-proBNP-108, irrespective of the type of heart failure. These results are consistent with the hypothesis that proBNP-108 is the major molecular form of BNP in the ventricle and that most of proBNP-108 is secreted from ventricle without proteolytic processing.

Yandle et al. (J. Clin. Endocrinol. Metab. 1993; 76: 832-8) previously analyzed the molecular forms of BNP in plasma taken from coronary sinus and peripheral vein in patients with heart failure. They showed that BNP-32 is the dominant form in coronary sinus, but proBNP-108 is the dominant molecular form in venous plasma. We also previously reported that proBNP-108, rather than BNP-32 is the dominant molecular form of BNP in normal subjects (Biochem. Biophys. Res. Commun. 1992; 185: 760-7). These observations raise the possibility that the proBNP-108 has a longer half-life in the circulation than BNP-32, probably due to different affinities of BNP-32 and proBNP-108 for their receptors. Indeed, the cGMP producing activity of BNP-32 is 10-20 fold higher than that of proBNP-108 in vascular smooth muscle cells and endothelial cells (J. Am. Coll. Cardiol. 2007; 49: 1071-8), suggesting that receptor-dependent metabolism of BNP-32 is higher than that of proBNP-108. Although we did not measure plasma BNP-32 or proBNP-108 in coronary sinus in this study, these results suggest that the metabolism of the two molecular forms of BNP is also an important determinant of the proBNP-108/total BNP ratio in peripheral circulation.

### Example 5

### Measuring the Post-Treatment or Natural Course of Plasma Concentrations of BNP-32 and proBNP-108 in Patients with Heart Failure

To investigate whether the pathophysiological status of heart failure affects the molecular form of BNP in plasma, we repeatedly measured plasma proBNP-108 and BNP-32 levels before and after the patients' conditions of heart failure had changed. We measured plasma proBNP-108 and BNP-32 in 5 patients with heart failure before and after their symptom had improved in response to treatments. We also measured plasma proBNP-108 and BNP-32 levels in 4 patients with heart failure before and after their symptom had deteriorated. Patients' characteristics are presented in Table 5.

TABLE 5
Clinical characteristics of patients whom condition
of heart failure improved or deteriorated

| | Improved | Deteriorated |
|---|---|---|
| Number | 5 | 4 |
| Sex (male/female) | (2/3) | (2/2) |
| Age | 72 ± 10 | 73 ± 9 |
| Etiology | | |
| IHD | 4 | 0 |
| ASD | 1 | 0 |
| MR | 0 | 2 |
| AS | 0 | 2 |

Values are mean ± S.D., IHD: ischemic heart disease, ASD: atrial septal defect, MR: mitral regurgitation, AS: aortic stenosis, MS: mitral stenosis,

### Blood Sampling

Blood samples (3 mL) were withdrawn from all subjects via the antecubital vein. Blood was immediately transferred into chilled glass tube containing disodium EDTA (1 mg/mL) and aprotinin (500 U/mL). Blood was centrifuged immediately at 4° C. and the plasma was frozen and stored at −80° C. until measurement.

Measurements of BNP-32 and proBNP-108 Concentrations

To calculate the proBNP-108/total BNP ratio, both plasma BNP-32 and plasma pro-BNP-108 concentration were measured in similar manner to Example 1.

### Statistical Analysis

All values (variations) are expressed as mean±SD. The statistical significance of differences between 2 groups was evaluated with Fisher's exact test or unpaired Student's t test, as appropriate. Log transformation was used to normalize the distribution of plasma peptide levels, if appropriate. Categorical variables were compared with the use of the chi-square test. Variables were compared among 3 groups by means of 1-way analysis of variance followed by Boneferoni's multiple comparison test. Correlation coefficients were calculated by linear regression analysis. P values of <0.05 were considered to indicate statistical significance.

### Results

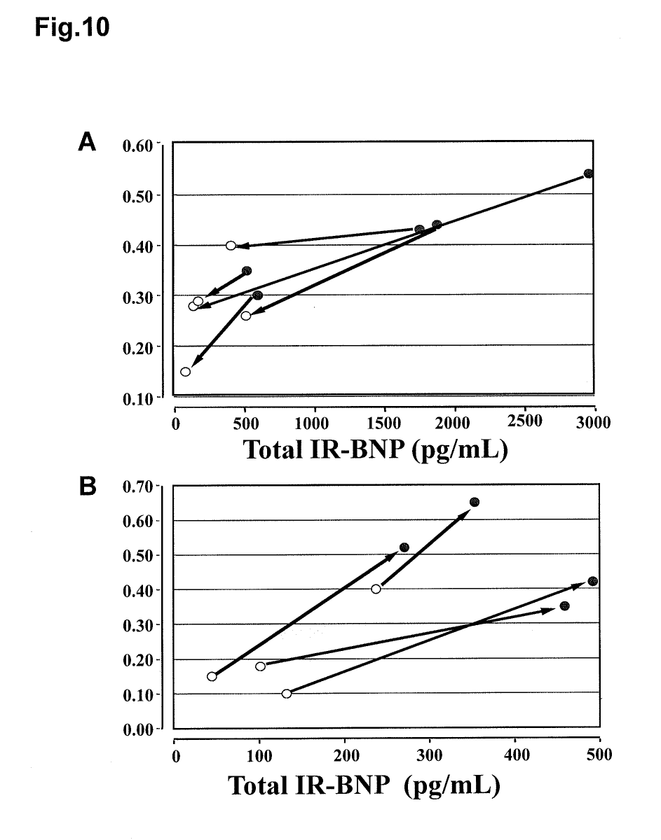

To investigate whether the pathophysiological status of heart failure affects the molecular form of BNP in plasma, we measured IR-BNP-32 and IR-proBNP-108 levels before and after treatments in patients with heart failure. As shown in FIG. 10-A, elevated plasma IR-BNP levels decreased after the treatments, accompanied by a reduction in the proBNP-108/total BNP ratio. In the cases that heart failure deteriorated during the observation, IR-BNP levels increased concomitantly with an increase in the proBNP-108/total BNP ratio (FIG. 10-B).

In decompensated heart failure, both IR-BNP-32 and IR-proBNP-108 were increased. In the patients whose conditions were improved by medical therapy, plasma IR-BNP-32 and IR-proBNP-108 decreased in association with a reduction in the proBNP-108/total BNP ratio. In contrast, when heart failure deteriorated, both plasma levels of IR-BNP-32 and IR-proBNP-108 were increased in association with an increase in proBNP-108/total BNP ratio. Thus, the proBNP-108/total BNP ratio is deduced to depend on the pathophysiological status of heart failure. An increased proBNP-108/total BNP ratio in severe heart failure may be explained in part by the increased production and secretion of proBNP-108 from the ventricle. Another possibility is that mRNA expression of proteolytic processing enzyme is not increased in parallel with the increase in mRNA expression of BNP precursor in severe heart failure. As a result, the proteolytic conversion of proBNP-108 into BNP-32 is thought to be reduced when proBNP-108 is secreted. A very recent study has shown that processing of proBNP-108 by furin is suppressed by O-glycosylation in the region close to the cleavage site (in printing). The regulatory mechanism in the O-glycosylation of proBNP-108 remain unknown at the present, upregulation of O-glycosylating enzyme in the failing myocardium may be associated with an increase in the plasma levels of proBNP-108 in the heart failure.

Based on the results of the present study, atrial overload increases production and secretion of IR-BNP mainly composed of BNP-32, while ventricular overload increases that of high MW IR-BNP corresponding to proBNP-108. As a result, the proBNP-108/total BNP ratio decreases in the atrial overload and increases in the ventricular overload, even though plasma IR-BNP levels is elevated in either case.

In summary, we analyzed the molecular forms of plasma BNP in heart failure by gel filtration HPLC and showed that not only IR-BNP-32, but also IR-proBNP-108 is present in the plasma of patients with control subjects and patients with atrial fibrillation and heart failure. The proBNP-108/total BNP ratio alters depending on the pathophysiological status of heart failure, which may be mainly related to atrial or ventricular overload. With the assay system currently used in the clinical setting, the BNP-32 kit cross-reacts with proBNP-108 at high ratios (Clin. Chem. 2007; 53: 866-73; Clin. Chim. Acta 2003; 334: 233-9; Clin. Chem. 2008; 54: 858-65; and Hypertension 2007; 50: e163), and this may be the reason for uncertainty and/or heterogeneity of BNP for the diagnosis of heart failure. Individual measurement of BNP-32 and proBNP-108 molecules may provide more useful information based on the causative mechanism to the cardiologist and general clinician who treats patients with heart failure.

Although certain preferred embodiments have been described herein, it should be understood that it is not intended that such embodiments be construed as limitations on the scope of the invention except as set forth in the appended claims. It should be understood that various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

SEQ ID NO: 1 is the amino acid sequence of the pre-pro-BNP (full-length), SEQ ID NO: 2 is the amino acid sequence of the pro-BNP, and SEQ ID NO:3 is the amino acid sequence of the BNP-32.

What is claimed is:

1. A method for determining the overload of either atrium or ventricle in a subject comprising the step of measuring proBNP-108 in a sample from the subject.

2. The method of claim 1 which further comprises the step of measuring BNP-32 in the sample from the subject.

3. The method of claim 1 which further comprises the step of estimating proBNP-108/total BNP in the sample from the subject.

4. The method of claim 1 wherein said determination or detection is directed to condition or progression of a disease selected from the group consisting of aortic stenosis, aortic regurgitation, mitral regurgitation, and atrial fibrillation.

5. A method for determining levels of progressing or treatment effect of heart failure comprising the step of measuring proBNP-108 in a sample from a subject.

6. The method of claim 5 which further comprises the step of measuring BNP-32 in the sample from the subject.

7. The method of claim 5 which further comprises the step of estimating proBNP-108/total BNP in the sample from the subject.

8. The method of claim 5 wherein said determination or detection is directed to one selected from the group consisting of staging of heart failure patient or its prognosis, determination of curative effect of beta blocking agent and ACE inhibitor for heart failure patient, grasp of dilated cardiomyopathy and determination of curative effect of dilated cardiomyopathy, grasp of myocardial infarction and grasp of remodeling after infarction, and typing of hypertrophic cardiomyopathy.

9. A kit for determining the overload of either atrium or ventricle in a subject comprising a substance that specifically binds to proBNP-108.

10. The kit of claim 9 wherein further comprises a substance that specifically binds to BNP-32.

11. The kit of claim 9 wherein the substance is an antibody.

12. The kit of claim 10 wherein the substance is an antibody.

13. A kit for determining levels of progressing or treatment effect of heart failure comprising a substance that specifically binds to proBNP-108.

14. The kit of claim 13 wherein the substance is an antibody.


SEQUENCE LISTING (3 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 32 aa, Homo sapiens
SPKMVQGSGCFGRKMDRISSSSGLGCKVLRRH